(19) United States
(12) Patent Application Publication
(10) Pub. No.: US 2006/0271137 A1
(43) Pub. Date: Nov. 30, 2006

(54) Apparatus and system to stimulate a nerve

(75) Inventor: Michael Stanton-Hicks, Chardon, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/385,399

(22) Filed: Mar. 21, 2006

Related U.S. Application Data
(60) Provisional application No. 60/684,210, filed on May 25, 2005.

(51) Int. Cl. A61N 1/05

(52) U.S. Cl. 607/118

(57) ABSTRACT

An electrode apparatus can includes an electrode body of a substantially flexible and non-conductive material, the electrode body having a generally cylindrical configuration with a diameter. At least a pair of electrodes along an inner surface of the electrode body are spaced axially apart from each other by a repeat distance that is functionally related to the diameter and that approximates a distance at which a given fascicle of a nerve, having the substantially the same diameter, periodically reconstitutes along an axial length of the nerve.

### RELATED APPLICATION

This application claims the benefit of U.S. Provisional Patent Application Ser. No. 60/684,210, which was filed on May 25, 2005, and entitled “Nerve stimulator,” which is incorporated herein by reference.

### TECHNICAL FIELD

The present invention relates generally a medical device and, more particularly, to an electrode apparatus and system for stimulating a nerve.

### BACKGROUND

Various types of stimulator devices have been developed for treatment for various conditions. For example, peripheral nerve stimulation (PNS) can provide a successful alternative therapy to patients experiencing chronic pain and who may be resistant to other treatment modalities. A candidate for PNS typically suffers from intractable pain that is secondary to nerve damage. The pain further may be isolated to a single nerve. PNS typically operates by stimulating a sensory nerve fiber to inhibit sensory nerve impulses from reaching a center of consciousness. By way of example, some common upper extremity nerves treated with PNS include the ulnar nerve, median nerve, and the radial nerve. Some lower extremity nerves that can be treated with PNS include the tibial nerve and the common peroneal nerve.

There are a number of recognized advantages associated with PNS. For example, a corresponding electrode can be implanted during a relatively simple surgical procedure. Additionally, after the electrode and pulse generator have been implanted in a patient, reasonably well-established testing processes can be employed to help tune the device to maximize the benefit for the patient. The testing process generally will vary according to the type of electrode and pulse generator being utilized and the desired electrical parameters for stimulation that is being delivered.

While various types and configurations of electrodes and stimulation methods have been developed, there exists a need for improved apparatus and method to stimulate a nerve.

### SUMMARY

The present invention relates generally to an electrode apparatus that can be used for providing electrical stimulation to a nerve. For example, the electrode apparatus can include at least a pair of electrodes that are spaced apart from each other an axial distance that anatomical relationship of the nerve that varies as a function of the diameter of the nerve to which the electrode apparatus is to be applied.

One aspect of the present invention provides an electrode apparatus that includes an electrode body of a substantially flexible and non-conductive material, the electrode body having a generally cylindrical configuration with a diameter. At least a pair of electrodes along are spaced axially apart from each other by a repeat distance that is functionally related to the diameter and that approximates a distance at which a given fascicle of a nerve, having the substantially the same diameter, periodically reconstitutes along an axial length of the nerve.

Another aspect of the present invention provides a stimulation system that includes an electrode body of a substantially flexible and non-conductive material, the electrode body having a generally cylindrical configuration with a diameter. A plurality of electrodes are along an inner surface of the electrode body. At least one pair of the electrodes is spaced axially apart from each other by a repeat distance that is functionally related to the diameter of the electrode body and that approximates a distance at which a given fascicle of a nerve, having the substantially the same diameter, periodically reconstitutes along an axial length of the nerve. A substantially resilient lead assembly extends outwardly from a substantially central location of the electrode body at an angle that is transverse relative to the exterior sidewall of the electrode body. A signal generator is electrically coupled to provide an electrical signal to the electrodes through lead wires that are attached to the electrode body by the lead assembly.

Still another aspect of the present invention provides an electrode apparatus that includes an electrode body of a substantially non-conductive material. The electrode body has a longitudinal sidewall that extends axially between spaced apart ends of the electrode body, the sidewall having an exterior sidewall portion and having an interior sidewall portion that defines a lumen dimensioned and configured for engaging a nerve. A plurality of electrodes are along the interior sidewall. A lead assembly extends longitudinally from a central portion of the exterior sidewall axially toward one of the ends of the electrode body to resiliently maintain a set of at least one lead wire substantially at a predetermined angle relative to the exterior sidewall portion of the electrode body.

### BRIEF DESCRIPTION OF THE DRAWINGS

FIG. 1 is an isometric view depicting an example of an electrode apparatus according to an aspect of the present invention.

FIG. 2 is a side elevation depicting the electrode apparatus of FIG. 1.

FIG. 3 depicts an example of another electrode apparatus according to an aspect of the present invention, in which the electrode body has been laid substantially flat.

FIG. 4 depicts an example of a stimulation system employing the electrode apparatus of FIG. 3 according to an aspect of the present invention.

FIG. 4A is a cross-sectional view taken along line 4A-4A in FIG. 4.

FIG. 4B is a cross-section view taken along line 4B-4B in FIG. 4.

FIG. 4C is a cross-sectional view taken along line 4C-4C in FIG. 4.

FIG. 5 depicts an example of another electrode apparatus according to an aspect of the present invention.

FIG. 6 is a cross-sectional view of the electrode apparatus taken along line 6-6 in FIG. 5.

FIG. 7 depicts an example of another electrode apparatus that can be implemented according to an aspect of the present invention.

FIG. 8 is a diagrammatic illustration of an electrode apparatus attached to a nerve bundle for treatment of a peripheral nerve of a patient's leg according to an aspect of the present invention.

### DETAILED DESCRIPTION

The present invention relates generally to an electrode apparatus that is dimensioned and configured according to the anatomy of a typical nerve. For example, the electrode apparatus includes at least a pair of electrodes that are spaced apart from each other an axial distance that is functionally related to the diameter of the nerve to which the electrode apparatus is to be applied. The predetermined distance between the pair of electrodes takes into account the anatomy of the nerve; namely, the periodically regrouping or reconstitution of a given fascicle of a nerve (corresponding to a bundle of nerve fibers also known as a funiculus) that tends to occur along the axial length of the nerve. As used herein, this periodic regrouping of a given nerve bundle along the axial length of a nerve is referred to herein as a “repeat length” or a “repeat distance.” As used herein, the repeat length (or distance) also encompasses integer multiples of the repeat length. As described herein, the repeat distance for a given nerve bundle varies as a function of the diameter of the nerve containing the given nerve bundle. Also the terms “nerve bundle” and “fascicle” are considered interchangeable with each other throughout this document, each corresponding to a myelinated and/or non-myelinated group of two or more nerve fibers.

Another aspect of the present invention relates to the orientation and placement of a lead wire assembly relative to the body of the electrode apparatus. For example, the lead wire assembly can extend from a central portion of the electrode body. Additionally or alternatively, the lead wire assembly can be oriented transversely relative to the exterior surface of the electrode body, such as may be angled proximally or distally relative to an end of the electrode body.

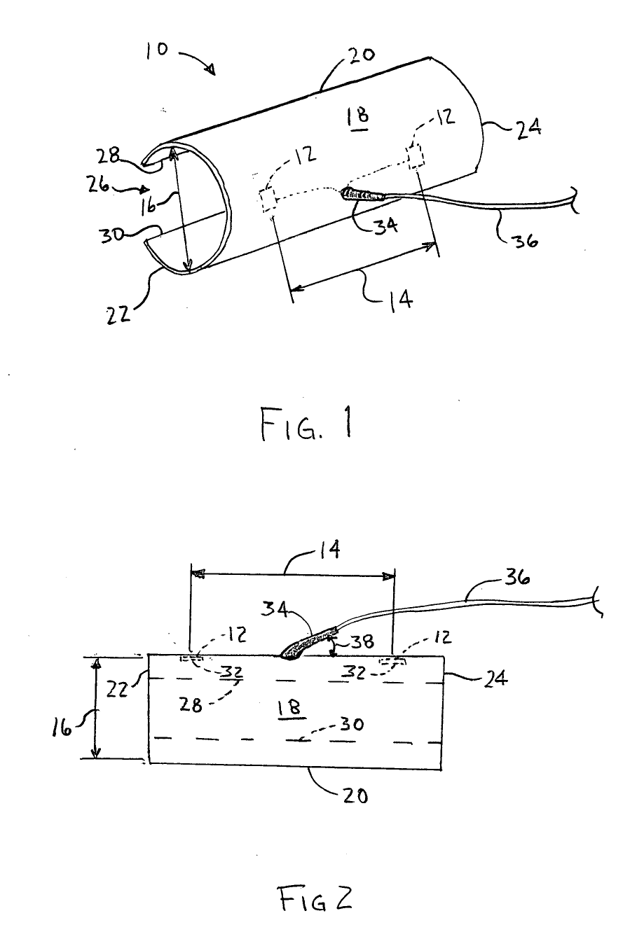

Turning now to the figures, FIGS. 1 and 2 depict different views of an electrode apparatus 10 that can be implemented according to an aspect of the present invention. The electrode apparatus 10 includes at least a pair of electrodes 12 that are axially spaced apart from each other a predetermined distance, indicated at 14. The predetermined distance 14 is established as a function of an inner diameter 16 of the electrode apparatus 10. The electrode apparatus 10 is configured so that the inner diameter 16 approximates the diameter of a target nerve to which the apparatus is to be applied. Accordingly, the distance 14 corresponds to the repeat distance associated with the target nerve, which that contains a plurality (e.g., two or more) nerve bundles, nerve fibers, or a combination of individual nerve fibers and bundles.

The electrode apparatus 10 can be employed for stimulation of one or more nerve fibers that may be contained within a peripheral nerve. As described herein, many peripheral nerves include a plurality of nerve fibers (and/or nerve bundles containing two or more nerve fibers). A given nerve bundle of a peripheral nerve can also contain motor fibers, sensory fibers and sympathetic fibers in varying numbers and combinations. The nerve bundles repeatedly unite (or reconstitute) and divide (or redistribute) and engage in plexus formation along the axial length of the nerve. The pattern changes in the internal structure of a nerve trunk along the length of a nerve can include variations in the number as well as the size of the nerve bundles. The regrouping and redistribution of component nerve bundles can rapidly change patterns, for example, transverse sections more than a few millimeters axially apart typically fail to present same pattern of component nerve fibers.

The regrouping and redistribution of component nerve bundles along the length of a nerve tends to result in a periodic regrouping or reconstitution of a given component nerve fiber at the repeat length. This periodic regrouping or reconstitution at the repeat length results the same component nerve bundle or a branch thereof returning to substantially the same angular and radial position when viewed in transverse sections of the nerve. The repeat length for a nerve varies as a function of the diameter of the nerve. For example, the repeat length can be expressed as follows:

$$L = 1 \frac{2}{3} \times D ,$$

where: L=the repeat length, and D=the diameter of the nerve.
   Therefore, by aligning one of the pair of electrodes 12 with a target nerve bundle or fiber, the same component nerve bundle should regroup and reconstitute at the other electrode of the pair if the pair of electrodes are spaced apart from each other by the repeat length 14. For example, assuming that a target nerve has a diameter of about 1.5 cm, the repeat length and spacing between the pair of electrodes 12 is about 2.5 cm.

The electrode apparatus 10 includes an elongated electrode body 18 of a substantially non-conductive (e.g., electrically insulating) and substantially biocompatible material. As depicted in the example of FIGS. 1 and 2, the electrode body 18 can have a generally cylindrical configuration having a sidewall 20 that extends between spaced apart ends 22 and 24. The sidewall 20 includes an interior sidewall portion that defines a lumen (or channel) dimensioned and configured for engaging an exterior surface of a target nerve. The electrode body 18 can also include a longitudinal opening 26. The opening 26 is defined by a pair of spaced apart edges 28 and 30 that extend axially (e.g., in a substantially parallel relationship) between the respective ends 22 and 24 of the sidewall 20. In the example of FIGS. 1 and 2, the electrode body has a substantially C-shaped cross-sectional configuration. The side edges 28 and 30 can be spaced apart from each other to provide a desired size opening to facilitate attachment of the electrode body 18 around a desired nerve, such as a peripheral nerve. For instance, the electrode body can have a cross-sectional configuration that extends approximately 270 degrees of a generally circular arc, with the opening forming the remaining 90 degrees of the circular arc.

The electrode body 18 can be considered a shell or wrapper that is formed of a substantially flexible, biocompatible material. The electrode body 18 can be deformed to facilitate mounting to a nerve. By way of example, the body 18 can include one or more sheets of a substantially flexible and electrically non-conductive material, such as Teflon, nylon, or Sylastic to name a few. The one or more sheets of material thus can be folded arcuately about a central longitudinal axis so that the side edges are spaced apart from each other to provide the generally cylindrical configuration shown in FIGS. 1 and 2. Those skilled in the art will understand and appreciate other substantially, substantially flexible and non-conductive biocompatible materials (e.g., including many elastomers or polymers) that can be utilized to provide the electrode body 18. By forming the electrode body of a sufficiently flexible material, the electrode body 18 can be further opened or deformed, such as by urging the side edges 28 and 30 apart to enlarge the opening 26. While the opening is enlarged, attachment of the electrode apparatus around a target nerve is facilitated. The flexible material utilized to provide the body 18 further can have shape memory (or elasticity), such as the Sylastic material identified above, such that the electrode body can return back to its original configuration after the deforming force is removed.

The electrodes 12 are formed of an electrically conductive material, such as can be formed as pads, thin sheets, or a thin layer or film of electrically conductive material. Examples of possible electrically conductive materials include aluminum, copper, and surgical steel, although other electrically conductive materials or alloys may be used. A radially inward contact surface 32 of each of the electrodes 12 can be exposed along the interior sidewall surface of the electrode body 18. The electrodes 12 can be attached to the interior sidewall surface of the electrode body 18. Alternatively, the electrodes 12 can be recessed partially into the interior sidewall surface of the electrode body 18. Those skilled in the art will understand and appreciate various approaches that can be utilized to secure the electrodes 12 to the electrode body. The particular type of structure or material (e.g., an adhesive, weld, fasteners, or sutures, or a combination thereof) may vary depending upon the material utilized to provide the electrode body 18 and the electrodes 12.

A lead assembly 34 extends from a central portion of the electrode body 18 between the ends 22 and 24. In the example of FIGS. 1 and 2, the lead assembly 34 is attached approximately one-half the distance between the respective ends 22 and 24. It will be understood and appreciated that the lead assembly 30 could extend from the body 18 at other axial locations, which may be nearer to either of the ends 22 or 24. In the example of FIG. 2, the lead assembly 34 is oriented at a predetermined angle, indicated at 38, relative to the exterior surface of the body 18. The angle 38, for example, may be at an angle that ranges from about 5 degrees to about 90 degrees (e.g., at about 45 degrees) relative to the surface of the body 18. Those skilled in the art will understand and appreciate that by orienting the resilient lead assembly 34 at an angle transverse to the electrode body 18 and extending from a generally central portion of the body, the amount of torque on the lead assembly 34 can be mitigated. The lead assembly 34 can be implemented as a substantially resilient structure that may be elastically deformable to retain its transverse orientation (the angle 38) relative to the exterior surface of the electrode body 18.

A corresponding set of one or more lead wires 36 extend from the lead assembly 34. The lead assembly 34 resilient secures the lead wires 36 relative to the electrode apparatus 10. The set of lead wires 36 electrically connect the respective electrodes 12 with a corresponding signal generator (not shown). For example, the signal generator can drive separate channels with electrical signal waveforms having desired electrical parameters (e.g., amplitude, frequency, phase symmetry, duty cycle, etc.). The signal generator can provide the electrical waveforms to energize each of the electrodes 12 over separate electrically conductive paths. Alternatively, the electrodes 12 can be electrically connected together and energized concurrently by such connection through the lead wires 36. Those skilled in the art will understand and appreciate various approaches that can be utilized to electrically connect the lead wires with the respective electrodes 12 including, for example, welding, adhesives, and soldering to name a few. While the example of FIGS. 1 and 2 shows a pair of electrodes 12 on the electrode apparatus 10, those skilled in the art will understand and appreciate that an electrode apparatus can include a greater number of electrodes according to an aspect of the present invention.

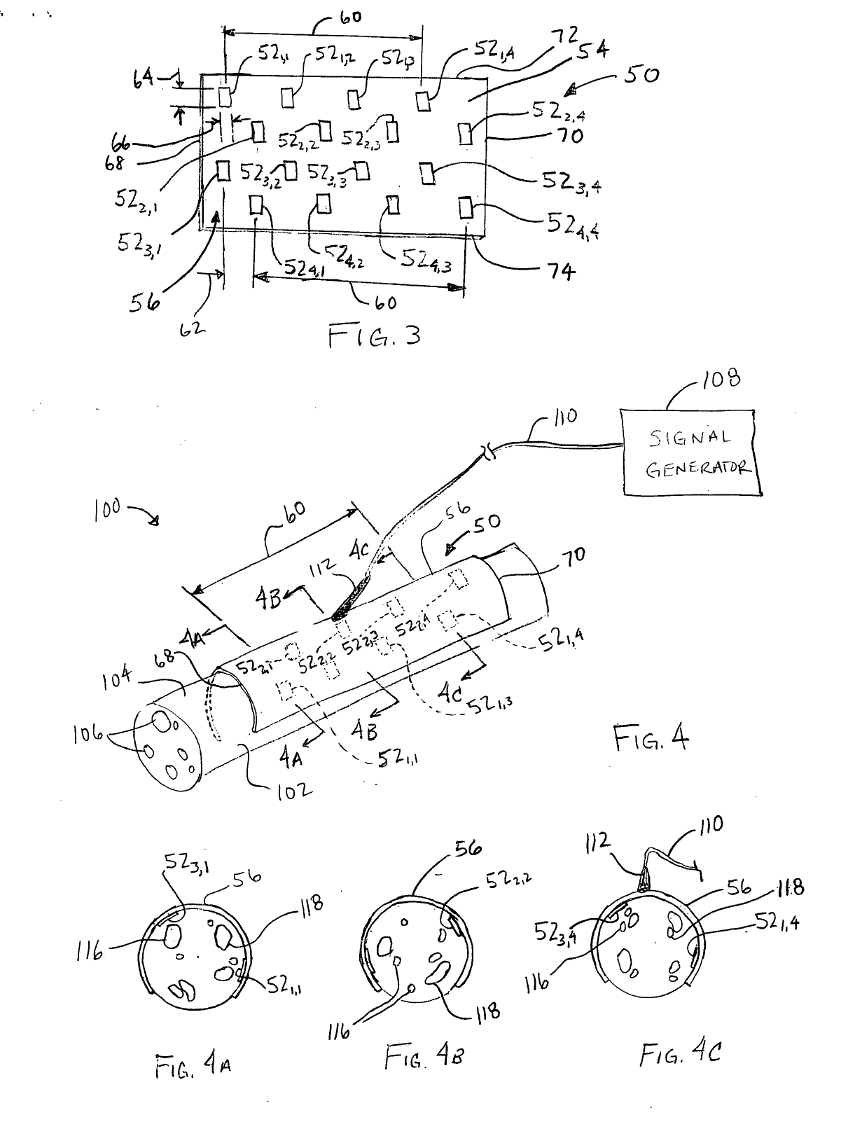

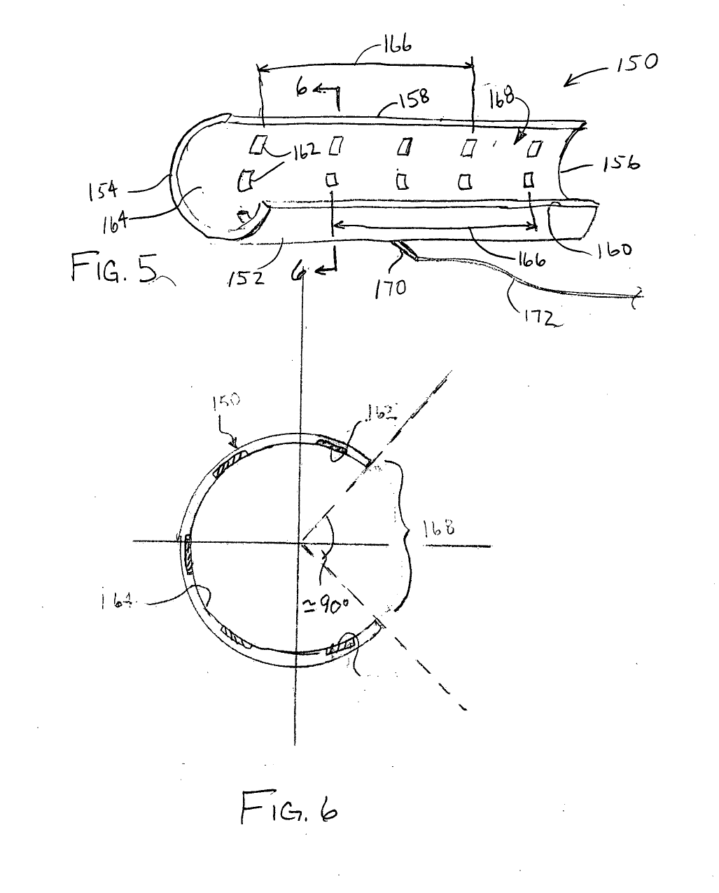

FIG. 3 depicts an example of another electrode apparatus 50 that can be implemented according to an aspect of the present invention. In the example of FIG. 3, the electrode apparatus 50 is shown in a substantially flat orientation to better illustrate the arrangement of electrodes. The electrode apparatus 50, however, can have a generally cylindrical (or C-shaped cross-sectional) configuration, such as shown and described herein. The electrodes 52 can be attached to the body 56 so as to be electrically exposed relative to the surface 54. The electrode apparatus 50 includes a plurality of electrodes 52 that are located along an interior surface 54 of an electrode body (or substrate) 56. The electrode body 56 can include one or more sheets of a substantially flexible material, such as described herein. The electrodes 52 can be attached to the body 56 so as to be electrically exposed relative to the surface 54. The electrode body 56 can have shape memory or otherwise be configured to return from the substantially flat orientation depicted in FIG. 3 to a corresponding generally cylindrical configuration (e.g., See FIG. 4).

For purposes of explanation, the electrodes 52 are considered arranged as a matrix of electrodes, in which subscripts associated with each of the respective electrodes identify a row and column of a two-dimensional electrode matrix. It is to be appreciated that the subscript nomenclature is used by way of example to designate a relative location of the electrodes 52, and is not utilized by way of limitation. In the example of FIG. 3, there are sixteen electrodes, depicted at electrode 52_{1,1} through electrode 52_{4,4}. The electrodes 52 are arranged on the surface 54 of the sheet 56 in a plurality (e.g., four) of rows and columns. The subscripts associated with each of the reference numbers of the electrodes 52 designate the row and column in the matrix for each electrode. Thus, there are four electrodes 52 in each of the four rows.

The electrodes 52_{1,1} and 52_{1,4} are spaced apart from each other by the repeat distance 60, as described herein. The electrodes 52 in each adjacent pair of rows are also offset from one another by an offset distance, indicated at 62 (e.g., for electrodes 52_{3,1} and 52_{4,4}). As an example, the offset distance 62 can be approximately one-half the axial distance between a pair of adjacent electrodes in the same given row. By offsetting electrodes in different rows, the ability to position a given electrode or multiple electrodes in alignment for stimulating a given nerve bundle with more than one electrode may be increased.

At least a pair of electrodes 52 in each of the respective rows is spaced apart from each other by the repeat distance 60. For example, in row 1, electrodes 52_{1,1} and 52_{1,4} are spaced apart by the repeat distance, and in row 3 electrodes 52_{3,1} and 52_{3,4} are spaced apart by the repeat distance. Similarly, in row 2, the electrodes 52_{2,1} and 52_{2,4} are spaced apart by the repeat distance 60 and, in row 4, the electrodes 52_{4,1} and 52_{4,4} are spaced apart from each other by the repeat distance.

In the example of FIG. 3, each of the electrodes 52 is dimensioned and configured to be substantially the same. As shown in FIG. 3, each of the electrodes 52 can have a circumferential dimension 64 that is greater than its axial dimension 66. For example, the circumferential dimension 64 may be approximately greater than about 1.5 times (e.g., about twice) the axial dimension 66. It is to be understood and appreciated that the electrodes 52 distributed on the electrode body 56 are not limited to electrodes that are the same size and configuration. Additionally, while the example electrodes 52 are shown as having a substantially rectangular contact surface, it will be understood that electrodes are not limited to such a configuration, as other shapes could be used, in certain embodiments, without departing from the teachings contained herein.

The electrode body 56 extends axially between spaced apart ends 68 and 70 thereof to define the respective sidewall 54 along which the electrodes 52 are disposed. The electrode body 56 also includes side edges 72 and 74 that extend between the respective ends 68 and 70 to define a respective opening when in the closed condition, such as shown in FIG. 4.

FIG. 4 depicts an example of a stimulator system 100 that includes an electrode apparatus 50 implemented according to an aspect of the present invention. For purposes of simplicity of explanation, and not by way of limitation, the electrode apparatus 50 corresponds to the apparatus of FIG. 3 described herein. Accordingly, in FIGS. 4, 4A, 4B and 4C the same reference numbers refer to parts and relationships between parts that were previously introduced with respect of FIG. 3.

In FIG. 4, the stimulation system 100 illustrates the electrode apparatus 50 applied to a nerve 102 in a circumscribing relationship. The nerve 102, for example, can be a peripheral nerve, such as in an upper extremity or a lower extremity. The nerve 102 has in its outermost epineurium 104 a plurality of nerve bundles 106 (i.e., fasicles or funiculi). Each of the nerve bundles 106 can include one or more motor fiber, sensory fiber, or sympathetic fibers in various numbers and combinations. The nerve 102 has a substantially cylindrical configuration along its length and a diameter. As mentioned above, the electrode body 56 also has similar generally cylindrical configuration and is dimensioned with a diameter that approximates the diameter of the nerve 102.

By way of example, for peripheral nerve stimulation, the electrodes 52 are aligned with and positioned adjacent to a sensory nerve bundle for providing electrical stimulation to prevent propagation of sensory nerve impulses along one or more respective sensory fibers. The electrical stimulation can be provided by a signal generator 108 that can be electrically coupled to the electrode apparatus 50 via corresponding lead wires 110. The lead wires 110 are attached to the electrode apparatus 50 and to the respective electrodes 52 via a lead assembly 112.

As discussed above with respect to FIG. 2, the lead assembly 112 can be formed of a substantially resilient material and be attached to a central portion of the electrode body 56. Additionally, the lead assembly 112 can be a longitudinal member that is oriented transversely relative to the exterior sidewall of the electrode body 56 (e.g., such as from an angle between 0 and 90 degrees). As depicted in FIG. 4, the lead assembly 112 is oriented axially relative to the electrode body 56 (angled toward the end 70) at approximately a 45-degree angle.

The signal generator 108 can be programmed and/or configured to provide electrical stimulation signals to one or more of the electrodes 52 having desired electrical parameters. For instance, a user or technician can define electrical parameters and the stimulator constructs a corresponding waveform. The electrical parameters can include amplitude, frequency, phase symmetry and duty cycle. The more complex the waveform, the more parameters are necessary to describe the waveform. Those skilled in the art will understand how to establish the parameters, for example, based on the condition being treated, the number of nerve fibers being stimulated as part of such treatment and the configuration of the electrode apparatus 50 (e.g., including the type number of electrodes 52).

FIGS. 4A, 4B, and 4C illustrate transverse cross sections of the electrode apparatus 50 and the nerve 102 taken at different axial positions. As mentioned above, the nerve 102 can include sensory nerve fibers as well as other types of nerve fibers (motor fibers and sympathetic fibers). In the cross-sectional view of FIG. 4A, a sensory nerve bundle 116 is located adjacent and aligned radially with the electrode 52_{3,1}. Due to branching and redistribution of the sensory nerve bundle 116, the nerve bundle can branch, redistribute and periodically regroup axially along the length of the nerve 102. Accordingly, in FIG. 4B, the sensory nerve bundle 116 has been divided into multiple branches that no longer reside at the same angular and radial position, as in the cross section of FIG. 4A. FIG. 4C is taken at the repeat distance 60 from the cross section of FIG. 4A. The periodic regrouping or alignment of the sensory nerve bundle 116 places the sensory nerve fibers adjacent the electrode 52_{3,4}, such as depicted in FIG. 4C. The signal generator 108 can electrically stimulate each of the electrodes 52_{3,1} and 52_{3,4} in a desired manner (e.g., concurrently or separately) to implement desired stimulation of the sensory bundle 116 and thereby inhibit sensory nerve impulses from traveling through the sensory nerve bundle. In FIGS. 4A, 4B and 4C another nerve bundle 118 may contain motor nerve fibers. Its redistribution and regrouping is also shown among the cross-sectional figures.

By providing the arrangements of electrodes having staggered electrodes between adjacent pairs of rows as shown in FIG. 3, and by having at least one pair of electrodes in each row spaced apart the repeat length, testing and training with the signal generator can be enhanced so as to improve the amount of electrical stimulation applied by the electrode apparatus 50. For example, since the arrangement of electrodes 52 usually provides at least a pair of electrodes that are aligned with a target nerve bundle (e.g., sensory nerve bundle 116), the signal generator 108 can provide a given aggregate current that is distributed across the respective electrodes. The distribution of the aggregate current enables a lower current that is applied to each of the electrode 52 relative to many existing approaches.

By providing lower current to each electrode for stimulating a given sensory bundle, the electrode apparatus 50 can deliver desired aggregate stimulation with a reduced likelihood of incidental stimulation of other (non-targeted) nerve fibers. This is contrast to the traditional approach for stimulating motor nerve fibers in which a larger current is applied to each electrode, which may result in inadvertent stimulation of non-targeted fibers. The electrode apparatus 50 thus can be utilized to block sensory nerve impulses to reduce pain for a patient while mitigating potential side effects relative to approaches that utilize existing electrode structures. The electrode apparatus 50 is not limited to stimulation of sensory nerve fibers or to peripheral nerve stimulation, as the approach described herein is equally applicable to other treatments, such as to stimulate motor fibers, for example.

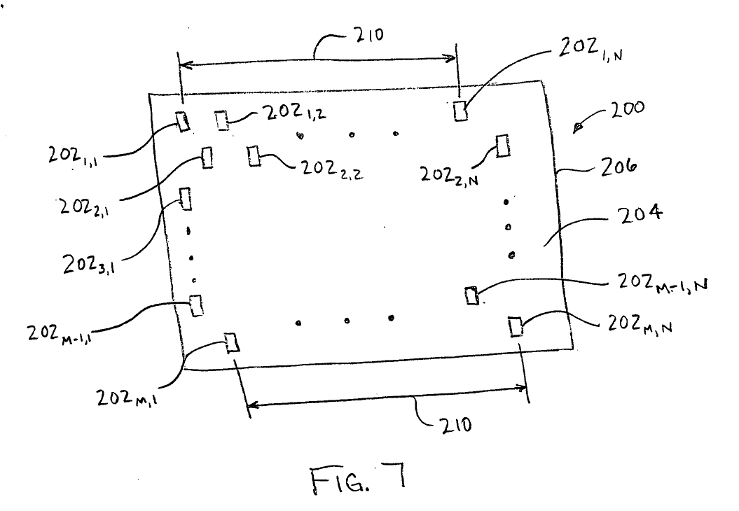

FIG. 5 depicts an example of another electrode apparatus 150 that can be implemented according to an aspect of the present invention. The electrode apparatus 150 includes an electrode body 152 that extends axially in a generally cylindrical arrangement between spaced apart ends 154 and 156. A longitudinal opening 168 extends between spaced apart side edges 158 and 160 to provide the electrode body 152 a substantially C-shaped cross section. A plurality of electrodes 162 are disposed along an inner surface 164, such as a plurality of rows of electrodes. Each electrode can be of an electrically conductive material, such as described herein.

In the example of FIG. 5, more than one pair of electrodes 162 are spaced axially apart from each other by a repeat length 166. For example, there can be two or more pairs of respective electrodes in a given row of electrodes that are spaced apart from each other by the repeat length 166. In this way, the initial placement of the electrode over a nerve (e.g., a peripheral nerve) has an increased likelihood of alignment with a desired type of nerve bundle for stimulation of corresponding nerve fibers (including branches thereof) at plural axial locations along the component nerve bundle.

FIG. 6 depicts a cross-sectional view of the apparatus 150 taken along line 6-6 in FIG. 5, further illustrating the C-shaped cross section. In the example of FIG. 6, it is shown that the arc of the C-shaped cylindrical body portion extends approximately 270 degrees thereby providing the opening 168 over the remaining approximately 90 degrees of the circular arc. By forming the electrode body 152 of a substantially flexible, electrically non-conductive material, such as described herein, the 90-degree opening 168 facilitates attachment over a desired nerve bundle.

The electrodes 162 are disposed circumferentially along the inner surface 164 of the electrode body 152. As an example, the radially inner surface (e.g., a contact surface) of the electrodes 162 is substantially flush with the inner surface 164 of the electrode body 152. For example, the electrodes 162 can be provided by recessing at least a portion of the respective electrodes in the electrode body 152, which may include one or more sheets of the electrically non-conductive material. For instance, the electrodes 162 may be sandwiched between superimposedly connected sheets of the flexible material. Additionally, after the electrode apparatus 150 has been attached over the desired peripheral nerve, the electrode body can be further attached to the nerve by one or more sutures (not shown).

Referring back to FIG. 5, the electrode apparatus 150 also include a lead assembly 170 that extends outwardly, transversely relative to the exterior sidewall of the electrode body 152. In the example, of FIG. 5, the lead assembly 170 is angled toward the end 156 of the electrode body, such as at about a 45-degree angle. Other angles can also be utilized, such as described herein. Lead wires 172, which may be encapsulated with an electrically non-conductive coating, extend further from the lead assembly 170. The lead wires 172 can extend from the electrode apparatus 150 in the same direction that the lead assembly is angled, such as to facilitate electrically connecting the electrodes with a signal generator (not shown). As described herein, the lead assembly 170 can be a substantially resilient material so as to substantially maintain its relative angular orientation relative to the sidewall, such as described herein. The lead assembly 170 may be implemented any structure or coating that secures the lead wires 172 to the electrode body 152 to helps maintain the desired orientation of the lead wires near the electrode body. The lead assembly 170, for example, may be capable of some elastic (or inelastic) deformation. The lead wires 172 can be connected with respective electrodes 162, such as by running the wires (which may be bare conductors) within an interior of the electrically insulating electrode body 152.

FIG. 7 depicts another example of an electrode apparatus 200 that includes a plurality of electrically conductive electrodes 202 disposed along a surface 204 of an electrode body 206. In FIG. 7, similar to FIG. 3, the electrode body 206 is shown in a substantially flat orientation. It is to be understood and appreciated that the electrode body 206 can have a generally cylindrical configuration, such as the substantially C-shaped cross section described herein. For example, the electrode body 206 can be a material having a shape memory to return to the C-shaped, generally cylindrical configuration or it may be formed into such a configuration during implantation.

In the example of FIG. 7, also similar to FIG. 3, the electrodes 202 are arranged as a two dimensional matrix having a plurality of M rows of electrodes and a plurality of N columns of electrodes, where M and N are positive integers denoting the number of electrodes in each row and column, respectively. Respective electrodes 202 are identified with subscripts that designate their placement by row and column. Thus, the electrode apparatus includes electrodes 202_{1,1} through 202_{M,N}, which provides for a total of M×N electrodes 202. The representation of FIG. 7 is intended to show that there can be any number of electrodes, which can be controlled by an associated signal generator. While the example of FIG. 7 shows the same number of N electrodes per row, it will be appreciated that different rows can include different numbers of electrodes, which further may be provided at different sizes.

The electrodes 202 can be independently controlled or selected sets of electrodes can be controlled concurrently by a common electrical connection with an output channel of a signal generator. For example, a set of electrodes in a given row that are spaced axially apart from each other at integer multiples of a repeat length 210 can be controlled (energized) concurrently. As described herein, the repeat length varies as a function of the diameter of the target nerve for the electrode apparatus. The particular number of rows and columns of electrodes and the number of electrodes in each row can vary according to the capabilities of the signal generator (not shown). For example, a 16-channel signal generator can be used to drive 16 electrodes independently or it can control more electrodes in which some are controlled concurrently by a common electrical connection with a given channel.

The particular arrangement and number of rows may vary from that shown and described herein. As technology improves, for example, more electrodes can be utilized with respective sets of two or more electrodes in a given axial row (e.g., rows 1 to N) being spaced apart integer multiples of the repeat length. Those skilled in the art will understand and appreciate various techniques that can be utilized to increase the number of channels that are capable of independently providing electrical stimulation to respective electrodes 202 that can be utilized. By providing at least two electrodes in a given axial row that are spaced apart from each other an integer multiple of the repeat distance, enhanced stimulation of nerve fibers can be implemented based on the teachings contained herein.

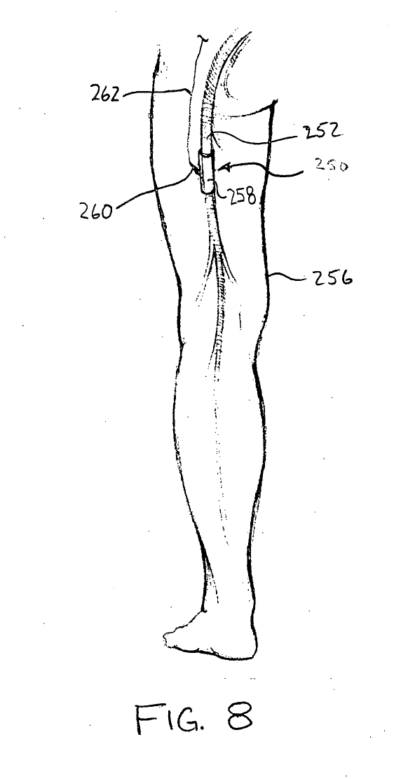

By way of further example, FIG. 8 depicts an example of an electrode apparatus 250 attached to a peripheral nerve 252 on a patient's leg 256. The electrode apparatus 250 can be any type of electrode apparatus implemented according to an aspect of the present invention based on the teachings contained herein. For example, the electrode apparatus 250 can include a plurality of electrodes in which at least a pair of electrodes are axially spaced apart from each other the repeat distance (or an integer multiple thereof) according to the diameter of the nerve 252. Additionally, the electrode apparatus 250 includes a lead assembly 258 that extends from a substantially central portion of the electrode body 258.

As shown in FIG. 8, the lead assembly 260 extends transversely and is angled proximally relative to electrode body 258 and the nerve 252. For example, the lead assembly 260 can be angled in the direction at which the core associated lead wires 262 extend for a connection to a corresponding signal generator, such as an implantable pulse generator (IPG) which is not shown in the example of FIG. 8. The angular orientation of the lead assembly 260 not only reduces torque associated with the respective lead assembly but also facilitates running the lead wires 262 between the electrode apparatus and the IPG. Those skilled in the art will understand and appreciate various types and configurations of IPGs or external signal generators that can be utilized. It should be understood and appreciated that the electrode apparatus can be flipped and oriented 180 degrees so that the lead assembly 260 would be angled axially oriented in the other direction (distally), such as if the IPG were located in the opposite direction along the length of the nerve 252.

While the example of FIG. 8 has illustrated the electrode apparatus 250 in a peripheral nerve of a patient's leg 256, those skilled in the art will understand and appreciate that use of an electrode apparatus according to an aspect of the present invention is not limited to treatment of a particular nerve. Additionally, while the above examples describe use of an electrode apparatus for peripheral nerve stimulation (PNS) of sensory fibers, it will be further appreciated that the electrode apparatus is equally applicable to stimulation of other types of nerve fibers, including, for example, motor fibers.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications, and variations that fall within the spirit and scope of the appended claims.

What is claimed is:

1. An electrode apparatus comprising:
an electrode body of a substantially flexible and non-conductive material, the electrode body having a generally cylindrical configuration with a diameter; and
at least a pair of electrodes along an inner surface of the electrode body, the pair of electrodes being spaced axially apart from each other by a repeat distance that is functionally related to the diameter and that approximates a distance at which a given fascicle of a nerve, having the substantially the same diameter, periodically reconstitutes along an axial length of the nerve.

2. The electrode apparatus of claim 1, wherein the repeat distance is defined as approximately
$$1 \frac{2}{3}$$
times an inner diameter of a cross section of the electrode body.

3. The electrode apparatus of claim 1, wherein the electrode body has a substantially C-shaped cross-section along the axial length thereof formed by an elongated sheet of the substantially flexible and non-conductive material that curves about a central longitudinal axis of the electrode body, side edges of the elongated sheet that extend axially between spaced apart ends of the sheet being spaced apart from each other to define a longitudinal opening.

4. The electrode apparatus of claim 1, wherein each of the electrodes has a circumferential dimension that is greater than an axial dimension thereof.

5. The electrode apparatus of claim 4, wherein the circumferential dimension of each electrode is greater than about 1.5 times the axial dimension.

6. The electrode apparatus of claim 1, wherein a plurality of electrodes are disposed in at least two axially extending rows of electrodes, the electrodes in one of the at least two rows being axially offset relative to the axial position of the electrodes in another of the at least two rows.

7. The electrode apparatus of claim 6, wherein at least one pair of electrodes in each of the at least two rows are spaced axially apart from each other by the repeat distance.

8. The electrode apparatus of claim 6, wherein the electrodes in each of the plurality of rows are spaced apart from adjacent electrodes in each respective row by a first axial distance, the axial offset being approximately one-half the first axial distance.

9. The electrode apparatus of claim 1, further comprising a substantially resilient lead assembly extending outwardly from a substantially central location of the electrode body at an angle that is transverse relative to the exterior sidewall of the electrode body.

10. The electrode apparatus of claim 10, wherein the angle is in a range from about five degrees to about 90 degrees relative to the exterior sidewall of the electrode body.

11. The electrode apparatus of claim 10, wherein the lead assembly extends axially from the electrode body toward one of axially spaced apart ends of the electrode body at an angle that is about 45 degrees relative to the exterior sidewall of the electrode body.

12. The electrode apparatus of claim 9, further comprising a signal generator that is electrically coupled to provide at least one electrical signal to the at least a pair of electrodes through a set of at least one lead wire that is attached to the electrode body by the lead assembly.

13. A stimulation system comprising:
an electrode body of a substantially flexible and non-conductive material, the electrode body having a generally cylindrical configuration with a diameter; and
a plurality of electrodes along an inner surface of the electrode body, at least one pair of the electrodes being spaced axially apart from each other by a repeat distance that is functionally related to the diameter of the electrode body and that approximates a distance at which a given fascicle of a nerve, having the substantially the same diameter, periodically reconstitutes along an axial length of the nerve;
a substantially resilient lead assembly extending outwardly from a substantially central location of the electrode body at an angle that is transverse relative to the exterior sidewall of the electrode body; and
a signal generator that is electrically coupled to provide an electrical signal to the electrodes through lead wires that are attached to the electrode body by the lead assembly.

14. The system of claim 13, wherein the electrode body has a substantially C-shaped cross-section along the axial length thereof formed by an elongated sheet of the substantially flexible and non-conductive material that curves about a central longitudinal axis of the electrode body, side edges of the elongated sheet that extend axially between spaced apart ends of the sheet being spaced apart from each other to define a longitudinal opening.

15. The system of claim 13, wherein each of the electrodes has a circumferential dimension that is greater than an axial dimension thereof.

16. The system of claim 13, wherein the plurality of electrodes are disposed axially in at least two rows of electrodes, the electrodes in one of the at least two rows being axially offset relative to the axial position of the electrodes in another of the at least two rows.

17. The system of claim 13, wherein the lead assembly extends longitudinally, axially toward one of the ends of the electrode body at an angle that is in a range from about five degrees to about 90 degrees relative to the exterior sidewall of the electrode body.

18. An electrode apparatus comprising:
an electrode body of a substantially non-conductive material, the electrode body having a longitudinal sidewall that extends axially between spaced apart ends of the electrode body, the sidewall having an exterior sidewall portion and having an interior sidewall portion that defines a lumen dimensioned and configured for engaging a nerve; and
a plurality of electrodes along the interior sidewall; and
a lead assembly that extends longitudinally from a central portion of the exterior sidewall axially toward one of the ends of the electrode body to resiliently maintain a set of at least one lead wire substantially at a predetermined angle relative to the exterior sidewall portion of the electrode body.

19. The apparatus of claim 18, wherein at least one pair of the plurality of electrodes is spaced axially apart from each other along the interior sidewall portion by a repeat distance that is functionally related to the diameter of the interior sidewall portion and that approximates a distance at which a given fascicle of a nerve, having the substantially the same diameter as the electrode body, periodically reconstitutes along an axial length of the nerve.

20. The apparatus of claim 19, wherein the plurality of electrodes are disposed axially in at least two axially arranged rows of electrodes, at least one pair of electrodes in each of the at least two rows being spaced axially apart from each other by the repeat distance.

21. The apparatus of claim 18, wherein the plurality of electrodes are disposed axially in at least two axially arranged rows of electrodes, the electrodes in one of the at least two rows being axially offset relative to the axial position of the electrodes in another of the at least two rows.

22. The apparatus of claim 18, wherein the lead assembly extends axially toward one of the ends of the electrode body at an angle that is in a range from about five degrees to about 90 degrees relative to the exterior sidewall portion of the electrode body.

23. The apparatus of claim 18, wherein the lead assembly extends axially toward one of the ends of the electrode body at an angle is about 45 degrees relative to the exterior sidewall portion of the electrode body.

24. The apparatus of claim 18, further comprising a signal generator that is electrically coupled to provide an electrical signal to the electrodes through a set of at least one lead wire that is connected to the electrode body by the lead assembly.
